(19) United States
(12) Patent Application Publication
(10) Pub. No.: US 2022/0016002 A1
(43) Pub. Date: Jan. 20, 2022

(54) NON-ALUMINIUM ANTIPERSPIRANT COMPOSITIONS

(71) Applicant: Conopco, Inc., d/b/a UNILEVER, Englewood Cliffs, NJ (US)

(72) Inventors: Philip Christopher Waterfield, Merseyside (GB); Aneliya Nikolova Zdravkova, Merseyside (GB)

(21) Appl. No.: 17/297,836

(22) PCT Filed: Oct. 23, 2019
(86) PCT No.: PCT/EP2019/078941

§ 371 Date: May 27, 2021

(30) Foreign Application Priority Data
Nov. 30, 2018 (EP) 18209520.8

(51) Int. Cl. A61K 8/34; A61Q 15/00; A61K 8/58; A61K 8/42; A61K 8/04

(52) U.S. Cl. CPC A61K 8/342; A61Q 15/00; A61K 8/345; A61K 2800/591; A61K 8/42; A61K 8/046; A61K 2800/30; A61K 8/585

(57) ABSTRACT

An antiperspirant composition comprising ethanol, amphiphilic material, volatile silicone and a humectant, the amphiphilic material being a mixture consisting of glycerol monolaurate and isostearyl alcohol at a ratio of from 25:75 to 45:55 by weight the composition having good stability and skin care credentials.

### FIELD OF INVENTION

The present invention is in the field of cosmetic compositions and their use as antiperspirants, in particular, non-aluminium antiperspirant compositions.

### BACKGROUND

EP 550,960 A1 (Unilever, 1992) discloses the use as an antiperspirant active of an amphiphilic material which forms, upon contact with perspiration, a water-insoluble liquid crystal phase of greater than one dimensional periodicity. This publication does not disclose ethanolic compositions, nor their stability issues.

WO 94/024993 (Unilever, 1994) discloses an antiperspirant composition comprising an amphiphilic material which forms, upon contact with perspiration, a water-insoluble liquid crystal phase of greater than one dimensional periodicity, in a cosmetic vehicle comprising a volatile silicone and containing less than 10% by weight of the total composition of a short chain monohydric alcohol.

### SUMMARY OF INVENTION

It is an object of the present invention to provide an antiperspirant composition that does not require the presence of an aluminium or zirconium salt to deliver an antiperspirancy benefit.

It is a further of the present invention to do this from a composition that has a high degree of storage stability and that delivers skin care benefits.

It is an object of the present invention to provide a base for an antiperspirant aerosol composition, the base being free from aluminium salts and having good stability.

In a first aspect of the invention, there is provided an antiperspirant composition comprising ethanol, amphiphilic material, volatile silicone and a humectant, the amphiphilic material being a mixture consisting of glycerol monolaurate and isostearyl alcohol at a ratio of from 25:75 to 45:, wherein:
(i) the ratio of amphiphilic material to (ethanol+volatile silicone) is from 1:9 to 2:3;
(ii) the ratio of volatile silicone to (ethanol+amphiphilic material) is from 1:10 to 11:9;
(iii) the ratio of ethanol to (amphiphilic material+volatile silicone) is from 3:7 to 3:2 and
(iv) the total amount of humectant in the composition is from 0.1 to 10%, excluding any propellant that may be present.

In a second aspect of the invention, there is provided a method of manufacture of an antiperspirant aerosol composition comprising the addition of propellant to a composition according to the first aspect of the invention.

In a third aspect of the invention, there is provided a cosmetic method of attaining an antiperspirant benefit comprising the topical application of a composition according to the first aspect of the invention.

### DETAILED DESCRIPTION

Herein, features expressed as “preferred” with regard to a particular aspect of the invention should be understood to be preferred with regard to each aspect of the invention (likewise, features expressed as “more preferred” or “most preferred”).

Herein, preferred features of the invention are particularly preferred when used in combination with other preferred features.

Herein, “ambient conditions” refers to 20° C. and 1 atmosphere pressure, unless otherwise indicated.

Herein, all percentages, ratios and amounts are by weight, unless otherwise indicated.

Herein, the word “comprising” is intended to mean “including” but not necessarily “consisting of”, i.e., it is non-exhaustive.

Herein, “cosmetic” methods and compositions should be understood to mean non-therapeutic methods and compositions, respectively.

Herein, an “amphiphilic material” is a material defined by having both hydrophilic and hydrophobic portions in its structure.

Herein, “water-insoluble” means having a solubility in water of less than 0.1% by weight (at 37° C.).

Antiperspirant compositions according to the present invention are preferably free of aluminium or zirconium antiperspirant salts. Indeed, they are more preferably free of any aluminium or zirconium salts.

Herein, “free of” means having less 0.1% and preferably less than 0.01% of the specified component or components.

Herein, a “volatile silicone” is a silicone having a vapour pressure of greater than 1 Pa at 25° C.

The compositions of the invention are particularly effectively when applied to the underarm regions of the human body and/or the feet. The compositions are especially effectively when applied to the underarm regions of the human body.

Antiperspirant aerosol compositions consist of a propellant and a base. The components of the base are typically mixed together first and the propellant is added last in a process sometimes called “gassing”. It is important that the base has good storage stability because there can be a significant period between the preparation of the base and the addition of the propellant.

Herein, the “base” of an antiperspirant aerosol composition is all the components of the total composition other than the propellant.

It is important that the fully formulated antiperspirant composition has good storage stability, so that it can survive prolonged transit to stores and extended periods on shelf prior to purchase and use.

### Amphiphilic Material

The amphiphilic material is a mixture consisting of glycerol monolaurate and isostearyl alcohol at a ratio of from 25 75 to 45:55

It is essential to have sufficient of the amphiphilic material present in the composition to achieve an acceptable degree of antiperspirancy when the composition is applied to the skin of the human body. The amphiphilic material is at least 10% of the tri-component mixture consisting of ethanol, amphiphilic material and volatile siloxane.

The ratio of amphiphilic material to (ethanol+volatile silicone) is from 1:9 to 2:3, this ratio being alternatively stated as being from 10:90 to 40:60. Hence, the amount of amphiphilic material in the tri-component mixture consisting of ethanol, amphiphilic material and volatile siloxane is from 10% to 40%.

The amphiphilic material serves as the antiperspirant active for the composition, just as in the prior publications EP 550,960 A1 and WO 94/024993 by Unilever.

According to a preferred embodiment of the invention, the amphiphilic material physically swells and forms a liquid crystal structure on contact with perspiration, hence enhancing the pore-blocking effect.

The content of the amphiphilic material in the total composition, ignoring any propellant therein, is preferably at least 10%, more preferably at least 12% and most preferably at least 15%.

### Volatile Silicone

The volatile silicone and its level of incorporation serves to reduce potential irritation and/or promote the good sensory properties of the composition.

The ratio of volatile silicone to (ethanol+amphiphilic material) in compositions of the invention is from 1:9 to 11:9, alternatively expressed as from 10:90 to 55:45. This equates to the volatile silicone being present at from 10% to 55% of the tri-component mixture consisting of ethanol, amphiphilic material and volatile siloxane. Preferably, the volatile silicone is present at from 10% to 50% and more preferably at from 15 to 40% of the tri-component mixture consisting of ethanol, amphiphilic material and volatile siloxane.

The content of volatile silicone in the total composition, ignoring propellant therein, is preferably from 10% to 67%, more preferably from 10% to 50% most preferably from 15% to 40%.

It is preferred that the volatile silicone comprises greater than 90% by weight of, or consists of siloxanes having from 2 to 6 silicone atoms, arranged in either a cyclic or linear fashion.

Linear siloxanes have the general formula: Me₃SiO(Me₂SiO)ₙSiMe₃, where Me=methyl group (—CH₃).
When n=0, the siloxane is hexamethyldisiloxane.
When n=1, the siloxane is octamethyltrisiiloxane.
When n=2, the siloxane is decamethyltetrasiloxane.
When n=3, the siloxane is dodecamethylpentasiloxane.

It is particularly preferred that the volatile silicone comprises greater than 90% by weight of, or consists of, siloxanes selected from the group consisting of hexamethyldisiloxane, octamethyltrisiiloxane, decamethyltetrasiloxane, dodecamethylpentasiloxane and decamethylcyclopentasiloxane.

It is especially preferred that the volatile silicone comprises greater than 90% by weight of, or consists of, siloxanes selected from the group consisting of hexamethyldisiloxane, octamethyltrisiiloxane, decamethyltetrasiloxane and decamethylcyclopentasiloxane.

### Ethanol

Ethanol is an essential component of compositions of the invention, serving to aid the solubilisation of the amphiphilic material.

The ratio of ethanol to (amphiphilic material+volatile silicone) is from 3:7 to 3:2, alternative expressed as from 30:70 to 60:40. This equates to ethanol being present at from 30 to 60% of the tri-component mixture consisting of ethanol, amphiphilic material and volatile silicone.

To further aid with the solubilisation of the amphiphilic material, it is preferred that the ratio of ethanol to amphiphilic material is at least 10:7. More preferably this ratio is at least 2:1 or 67:33.

The content of ethanol in the total composition, ignoring any propellant therein, is preferably at least 30%, more preferably at least 33% and most preferably at least 35%.

### Humectant

A humectant is an essential component of compositions od the invention. Humectants used in the present invention are hygroscopic materials having the ability to form hydrogen-bonds.

The purpose of the humectant is to aid skin care by retaining moisture in the skin. This can improve the barrier properties of the skin.

Preferred humectants for use in the present invention have multiple hydrophilic groups, in particular hydroxyl groups and/or amine groups. Such materials can be particularly effective hydrogen-bond donors making them particular good humectants.

Herein, amine groups include such groups linked to a carbonyl group to form an amide, whether part of larger functional group or not.

Particularly preferred humectants are selected from glycerol, 2-hydroxyethyl urea (HEU) and propylene glycol (PG).

Further preferred humectants are polyhydroxylated materials like PG, dipropylene glycol (DPG) and glycerol.

An especially preferred humectant is glycerol.

The total amount of humectant used is from 0.1 to 10%, excluding any propellant that may be present. This level is preferably from 0.5 to 6% and more preferably from 1 to 5% of the composition excluding any propellant that may be present.

Humectants have been difficult to incorporate into conventional antiperspirant compositions comprising aluminium and zirconium antiperspirant salts. We have found that they can be incorporated into the alternative antiperspirant compositions of the present invention by careful selection of the amounts and ratios of components. Without such selection, multiphasic or unstable compositions result.

### Other Components

In preferred embodiments of the invention, the composition is an antiperspirant aerosol composition comprising a propellant. In such compositions, the propellant preferably comprises from 35 to 95% of the total composition, more preferably from 40 to 90% and most preferably from 50 to 85% of the total composition.

When employed, the propellant is commonly either a compressed gas or a material that boils at below ambient temperature, preferably at below 0° C., and especially at below −10° C. Examples of compressed gasses include nitrogen and carbon dioxide. Examples of low boiling point materials include dimethyl ether. Other possible low boiling point materials that may be used as propellants are hydrofluorocarbons containing from 2 to 4 carbons, at least one hydrogen and 3 to 7 fluorine atoms.

In certain preferred embodiments involving antiperspirant aerosol compositions, the propellant used comprises dimethyl ether or hydrocarbon.

When compositions according to the invention are aerosol compositions, they can be made in a conventional manner by first preparing a base composition, charging the base composition into the aerosol can, fitting a valve assembly into the mouth of the can, thereby sealing the can, and thereafter charging propellant into the can to a desired pressure, and finally fitting an actuator on or over the valve assembly.

A preferred additional component of compositions of the invention is a deodorant active. These are typically antimicrobial agents active against bacterial on the skin of the human body. These serve to reduce malodour and especially useful in compositions in which the amphiphilic material is not itself an antimicrobial agent.

When employed, the level of incorporation is preferably 0.01%-5%, more preferably from 0.01-2% and most preferably from 0.03%-0.5% by weight of the total composition.

Preferred anti-microbial deodorant agents are those that are more efficacious than simple alcohols such as ethanol. Particularly preferred anti-microbial deodorant agents are soluble in ethanol, meaning that they a solubility in ethanol of at least 10 g/L at 20° C.

Examples of suitable anti-microbial deodorant agents include niacinamide; quaternary ammonium compounds, like cetyltrimethylammonium salts; chlorhexidine and salts thereof; and diglycerol monocaprate, diglycerol monolaurate, glycerol monolaurate, and similar materials, as described in “Deodorant Ingredients”, S. A. Makin and M. R. Lowry, in “Antiperspirants and Deodorants”, Ed. K. Laden (1999, Marcel Dekker, New York). More preferred are polyhexamethylene biguanide salts (also known as polyaminopropyl biguanide salts), an example being Cosmocil CQ available from Arch Chemicals, 2′,4,4′-trichloro,2-hydroxy-diphenyl ether (triclosan), 3,7,11-trimethyldodeca-2,6,10-trienol (farnesol), essential oils such as Tea Tree Oil and Thyme Oil, climbazole, octapyrox, ketoconazole, zinc pyrithione and mixtures thereof.

A preferred optional component is a preservative, such as ethyl or methyl parabens or BHT (butyl hydroxy toluene), typically in an amount of from 0.01 to 0.1% by weight of the total composition.

The invention will now be described by some examples, which do not limit the extent of the invention.

### EXAMPLES

In the following examples, examples according to the invention are indicated by numbers and comparative examples are indicated by upper case letters.

The volatile silicones used in these examples were:

DC245=cyclopentasiloxane and
DC200 (1.5 cS)=decamethyltetrasiloxane, herein referred to as DC200.
Each of these materials is available from Dow Corning.

The amphiphilic material used in these examples is designated as “lipid” and was a 60:40 blend of glycerol monolaurate and isostearyl alcohol.

The ethanol used in these examples was absolute alcohol.

The volatile silicone used in these examples was either DC200 or a 15:85 mix of DC245 and DC200, referred to in Table 1 as “Mix”.

The humectants used in these examples were hydroxyethyl urea (HEU), propylene glycol (PG) and glycerol.

The compositions indicated in Table 1 were prepared by methods known in the art.

TABLE 1

| | Ethanol | Volatile silicone | | Lipid | Humectant | |
|---|---|---|---|---|---|---|
| Example | % w/w | Nature | % w/w | % w/w | Nature | % w/w |
| 1 | 45 | Mix | 39 | 15 | HEU | 1 |
| 2 | 45 | DC200 | 39 | 15 | HEU | 1 |
| 3 | 46 | Mix | 33 | 20 | HEU | 1 |
| 4 | 46 | DC200 | 33 | 20 | HEU | 1 |
| 5 | 44 | Mix | 37 | 15 | PG | 4 |
| 6 | 44 | DC200 | 37 | 15 | PG | 4 |
| 7 | 45 | Mix | 21 | 20 | PG | 4 |
| 8 | 45 | DC200 | 21 | 20 | PG | 4 |
| 9 | 60 | DC200 | 11 | 25 | PG | 4 |
| 10 | 50 | DC200 | 11 | 35 | PG | 4 |
| A | 30 | DC200 | 21 | 45 | PG | 4 |
| B | 25 | DC200 | 46 | 25 | PG | 4 |
| 11 | 35 | DC200 | 51 | 10 | PG | 4 |
| 12 | 44 | Mix | 37 | 15 | Glycerol | 4 |
| 13 | 44 | DC200 | 37 | 15 | Glycerol | 4 |
| 14 | 45 | Mix | 21 | 20 | Glycerol | 4 |
| 15 | 45 | DC200 | 21 | 20 | Glycerol | 4 |
| 16 | 60 | DC200 | 11 | 25 | Glycerol | 4 |
| 17 | 50 | DC200 | 11 | 35 | Glycerol | 4 |
| C | 30 | DC200 | 21 | 45 | Glycerol | 4 |
| D | 25 | DC200 | 46 | 25 | Glycerol | 4 |
| 18 | 35 | DC200 | 51 | 10 | Glycerol | 4 |

The stability of the examples 5 to 8 and 12 to 15 was monitored for 8 weeks at 20° C. The stability of all of the other examples and comparative examples was monitored for 4 weeks at ambient temperature.

All of the examples according to the invention proved to be stable over the period tested. Comparative examples A, B, C and D proved to be instable. Instability manifested itself by phase separation, the lipid component(s) typically crystallising out of the liquid phase.

1. An antiperspirant composition comprising:
ethanol;
amphiphilic material;
volatile silicone having a vapour pressure of greater than 1 Pa at 25° C.; and a humectant,
wherein the amphiphilic material is a mixture consisting of glycerol monolaurate and isostearyl alcohol at a ratio of from 25:75 to 45:55 by weight wherein:
(i) the ratio of amphiphilic material to ethanol+volatile silicone is from 1:9 to 2:3 by weight;
(ii) the ratio of volatile silicone to ethanol+amphiphilic material is from 1:10 to 11:9 by weight;
(iii) the ratio of ethanol to amphiphilic material+volatile silicone is from 3:7 to 3:2 by weight; and
(iv) the total amount of humectant in the composition is from 0.1 to 10% by weight, excluding any propellant that may be present.

2. A composition according to claim 1, wherein the composition is an aerosol composition comprising a propellant.

3. A composition according to claim 2, wherein the propellant is dimethyl ether (DME) or a hydrocarbon.

4. A composition according to claim 1, wherein the propellant is from 30 to 90% by weight of the composition.

5. A composition according to claim 1, wherein the volatile silicone comprises greater than 90% by weight of siloxanes selected from the group consisting of hexamethyldisiloxane, octamethyltrisiiloxane, decamethyltetrasiloxane, dodecamethylpentasiloxane and decamethylcyclopentasiloxane.

6. A composition according to claim 1, wherein the volatile silicone comprises greater than 90% by weight of siloxanes selected from the group consisting of hexamethyldisiloxane, octamethyltrisiiloxane, decamethyltetrasiloxane and decamethylcy cl op entasil oxane.

7. A composition according to claim 1, which is free from aluminium or zirconium antiperspirant salts.

8. A composition according to claim 1, wherein the humectant has multiple hydroxyl groups and/or amine groups.

9. A composition according to claim 1, wherein the humectant is selected from glycerol, 2-hydroxyethyl urea (HEU) and propylene glycol (PG).
